(19) United States
(12) Patent Application Publication
(10) Pub. No.: US 2002/0045241 A1
(43) Pub. Date: Apr. 18, 2002

(54) T CELLS SPECIFIC FOR KIDNEY CARCINOMA

(76) Inventor: DOLORES J. SCHENDEL, MUNCHEN (DE)

Correspondence Address: ARENT FOX KINTNER PLOTKIN & KAHN, 1050 CONNECTICUT AVENUE, SUITE 600, WASHINGTON, DC, 20036-5339, US

(21) Appl. No.: 08/881,509

(22) Filed: Jun. 24, 1997

(30) Foreign Application Priority Data
Jun. 24, 1996 (DE) 196 25 191.5

(51) Int. Cl. C12N 15/00; C12N 15/74; C12N 15/63; C12N 15/09; C07H 21/04; C12N 1/22; C12N 15/70

(52) U.S. Cl. 435/252; 435/320.1; 536/23.53

(57) ABSTRACT

The present invention concerns new nucleic acid and amino acid sequences of the human T cell receptor and their use for the diagnosis and therapy of carcinomas in particular of kidney cell carcinomas.

### DESCRIPTION

The present invention concerns new nucleic acid and amino acid sequences of the human T cell receptor and their use for the diagnosis and therapy of carcinomas in particular of kidney cell carcinomas.

The T lymphocytes of the immune system are responsible for the cellular immune response. They are able to recognize and eliminate diseased body cells, e.g. cells which contain foreign proteins, or tumour cells. Diseased body cells are recognized by the so-called T cell receptor (TCR) which binds an antigen in the form of short peptide fragments which is specific for the diseased cell. These peptide fragments are presented by MHC molecules on the cell surface.

T cell receptors are composed of two different polypeptide subunits, usually the so-called T cell receptor α or β chains which are linked together by a disulfide bridge. The α and β chains are in turn composed of variable and constant regions. The variable regions of the a chain comprise V and J gene segments and the variable regions of the β chain comprise V, D and J gene segments.

The TCR α chain gene is composed of over 100 variable segments each of which contains an exon for a V region in front of which there is another exon which codes for a leader sequence which enables transport of the protein to the cell surface. A group of 61 J segments lies at a considerable distance from the V segments. The J segments are followed by a single C segment for the constant region which in turn contains separate exons for the constant region and the hinge region as well as an exon for the transmembrane and cytoplasm regions.

The TCR β chain gene contains a group of approximately 30 V gene segments which are at some distance from 2 separate clusters which each contain a single D segment and 6 or 7 J segments as well as a single C segment. Each constant segment of the β chain has separate exons for the constant, the hinge, the transmembrane and the cytoplasm region.

During the development of the T cell the separate segments are linked by somatic recombination. In the case of the a chain a Vα gene segment gets next to a Jα gene segment and hence a functional exon is formed. Transcription and splicing of the VJα exon to the constant region leads to the formation of the mRNA which is translated into the TCR α chain. The rearrangement of the Vβ, Dβ and Jβ gene segments coding for the variable domain of the β chain creates a functional exon which is transcribed and attached to Cβ by splicing. The mRNA which forms is translated into the TCR β chain. The α and β chains join together after their biosynthesis to form an α: β TCR heterodimer. The highly variable region of the TCR which is responsible for the specificity of antigen recognition and is located in the linkage region of the V, (D) and J gene segments is referred to as the CDR3 region.

Due to the high variability of T cell receptors it is very time-consuming to identify specific nucleotide and amino acid sequences in particular in the area of the CDR3 antigen recognition region. There is therefore a great need to provide nucleic acid and amino acid sequences of T cell receptors which are able to specifically recognize clinically relevant peptide antigens in particular tumour-specific peptide antigens.

According to the invention tumour-infiltrating lymphocytes (TIL) could be isolated from a kidney carcinoma which have a high specificity for tumour tissue from patients with the HLA-A*0201 allele. These TIL show no reaction with healthy kidney tissue from the same patient.

An analysis was carried out of the nucleotide and amino acid sequences of the T cell receptors expressed by these TIL. In this process a homogeneous CD8⁺T cell clone was firstly obtained by culturing and periodic restimulating the TIL over a period of 62 and 74 days respectively. The cDNA coding for the α and β chain of the T cell receptor was sequenced. The nucleotide and amino acid sequence of the α chain are shown in the sequence protocols SEQ ID NO. 1 and SEQ ID NO. 2. The CDR3α region in SEQ ID NO. 1 extends from bp 313 to 348 corresponding to the amino acids 87-98 in SEQ ID NO. 2. The nucleotide and amino acid sequence of the β chain are shown in the sequence protocols SEQ ID NO. 3 and SEQ ID NO. 4. The CDR3β region in SEQ ID NO. 3 extends from bp 331 to 369 in SEQ ID NO. 3 corresponding to the amino acids 90-102.

In the case of the α chain a combination of Vα20 with Jα22 was found in the variable region and in the case of the β chain a combination of Vβ22, Dβ2 and Jβ2.7.

Subsequently a sequence analysis of the tumour-specific T cell receptors was carried out with a culture for only 24 days. In this case a homogeneous T cell clone was not found but rather a mixture of several T cell species. The amino acid sequence shown in SEQ ID NO. 2 as well as in all two further amino acid sequences were able to be identified for the α chain. 11 out of 56 examined T cell species coded for the amino acid sequence shown in SEQ ID NO. 2 of the CDR3α region from position 87 to 98. the nucleotide sequence of the a chains in these T cells differed from the sequence shown in SEQ ID NO. 1 only by a substitution of T by G at position 324.

The nucleotide and amino acid sequence of the CDR3 region of a further α chain which was identified in 38 out of the 56 examined T cells is shown in the sequence protocols SEQ ID NO. 5 and 6. In addition two further T cell species were identified which contained a CDR3α region with the same amino acid sequence to that shown in SEQ ID NO. 6 but whose nucleotide sequence each differed by a base substitution (C at position 9 substituted by G or T at position 12 substituted by C).

The nucleotide and amino acid sequence of the CDR3α region from a third T cell variant which occurred at a frequency of 5 out of 56 examined T cell species is shown in the sequence protocols SEQ ID NO. 7 and 8.

The corresponding sequencing of the β chains yielded a total of 6 different amino acid sequences for the CDR3 region. A CDR3β sequence which was found in 15 out of 50 examined T cells is shown in the sequence protocols SEQ ID NO. 9 and 10. A further T cell species contained the same amino acid sequence but a different nucleotide sequence (substitution of A at position 15 by T).

One T cell species in each case contained the nucleotide and amino acid sequences shown in the sequence protocols SEQ ID NO. 11 and 12, 13 and 14 or 15 and 16 in the CDR3β region.

27 out of 50 clones contained the nucleotide and amino acid sequences shown in the sequence protocols 17 and 18 in the CDR3β region. 4 out of 50 examined clones contained the nucleotide and amino acid sequences shown in the sequence protocols SEQ ID NO. 19 and 20 in the CDR3β region.

In addition an in situ sequencing of TIL was carried out i.e. a sequencing without prior culture. For this the entire RNA was isolated from the tumour, a TCR-specific cDNA was prepared using a TCRα- or TCRβ-specific primer and reverse transcriptase and this cDNA was selectively amplified by PCR using family-specific primers (Vα20 and Vβ22). The amplification products were cloned into E. coli and sequenced. In this process a series of single sequences was obtained.

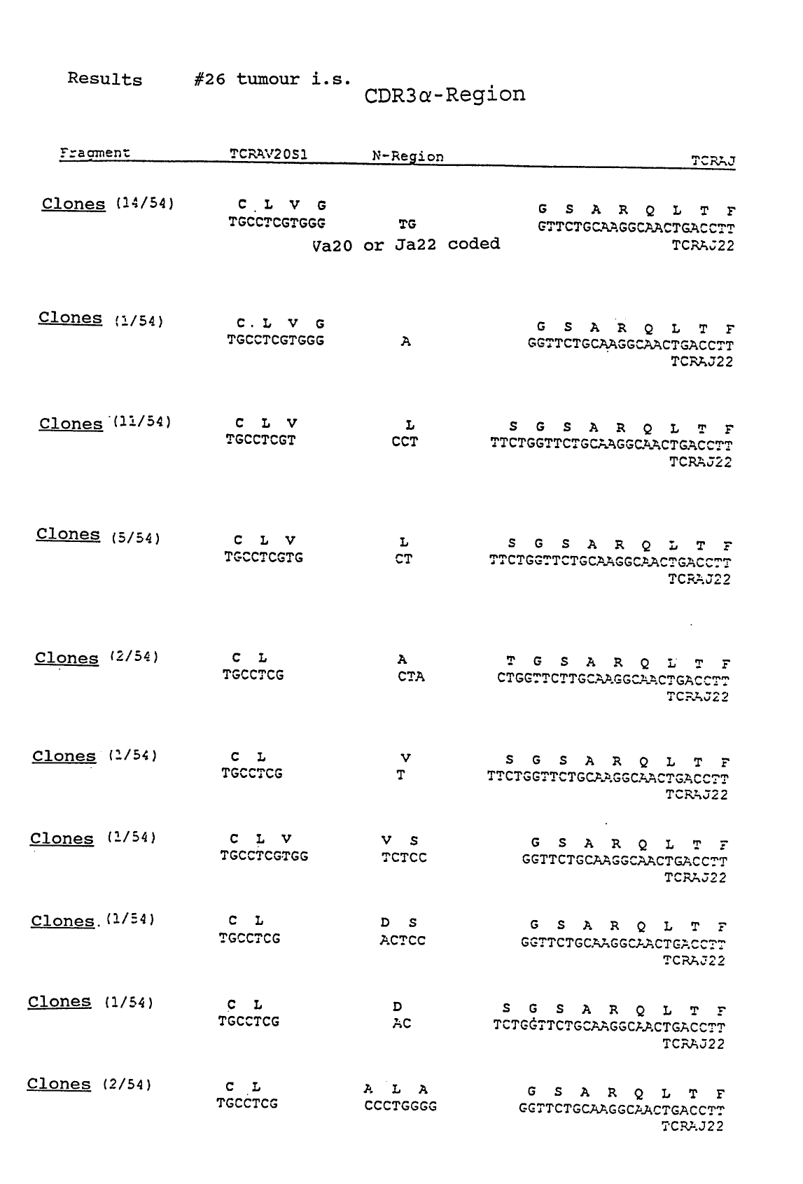

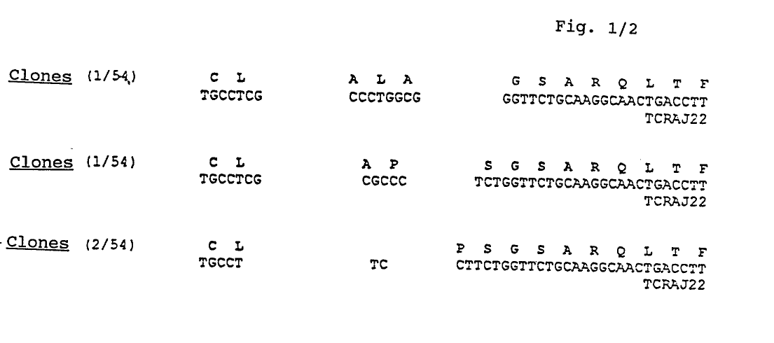

Circa 60% of all sequences of the a chain correspond to the amino acid sequences shown in the sequence protocols SEQ ID NO. 2, 6 and 8. A further 20% had very similar sequences which were also composed of a combination of Vα20 and Jα22. An overview of the CDR3α regions identified in this in situ sequencing of T cells from patient 26 is shown in FIG. 1.

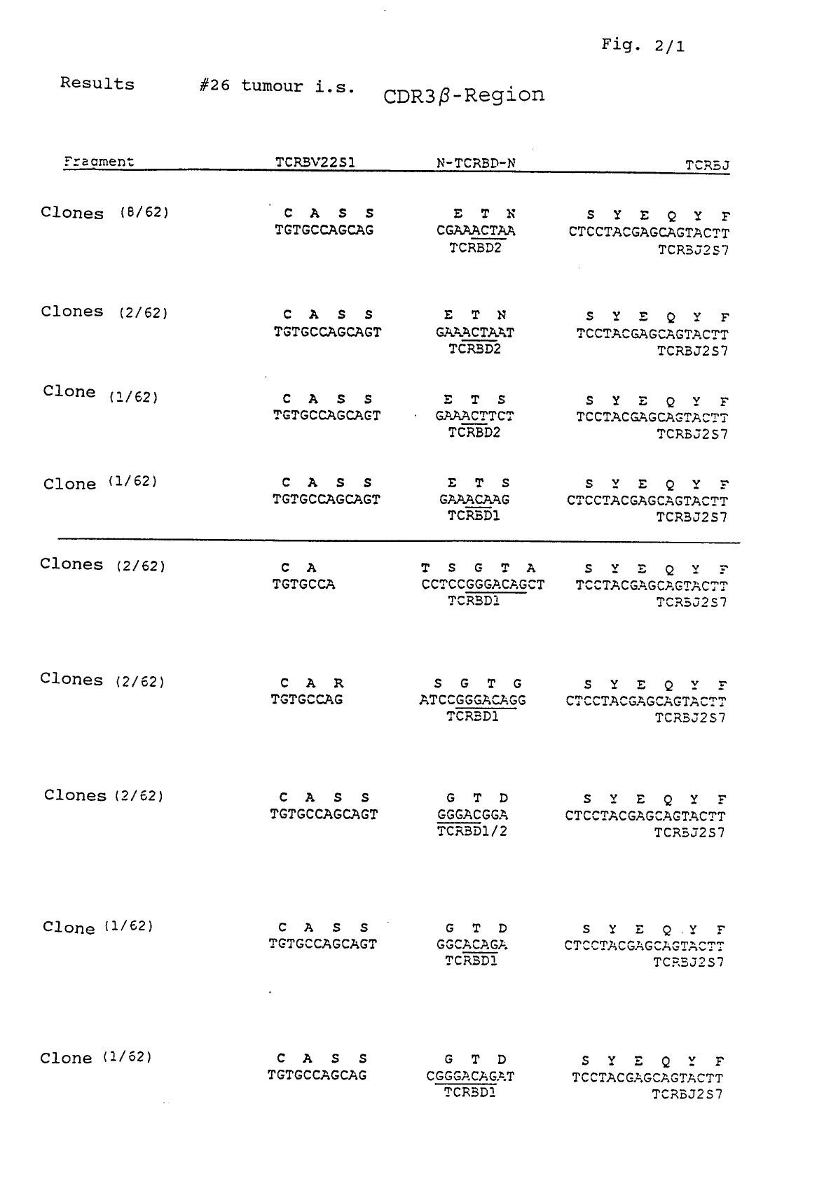

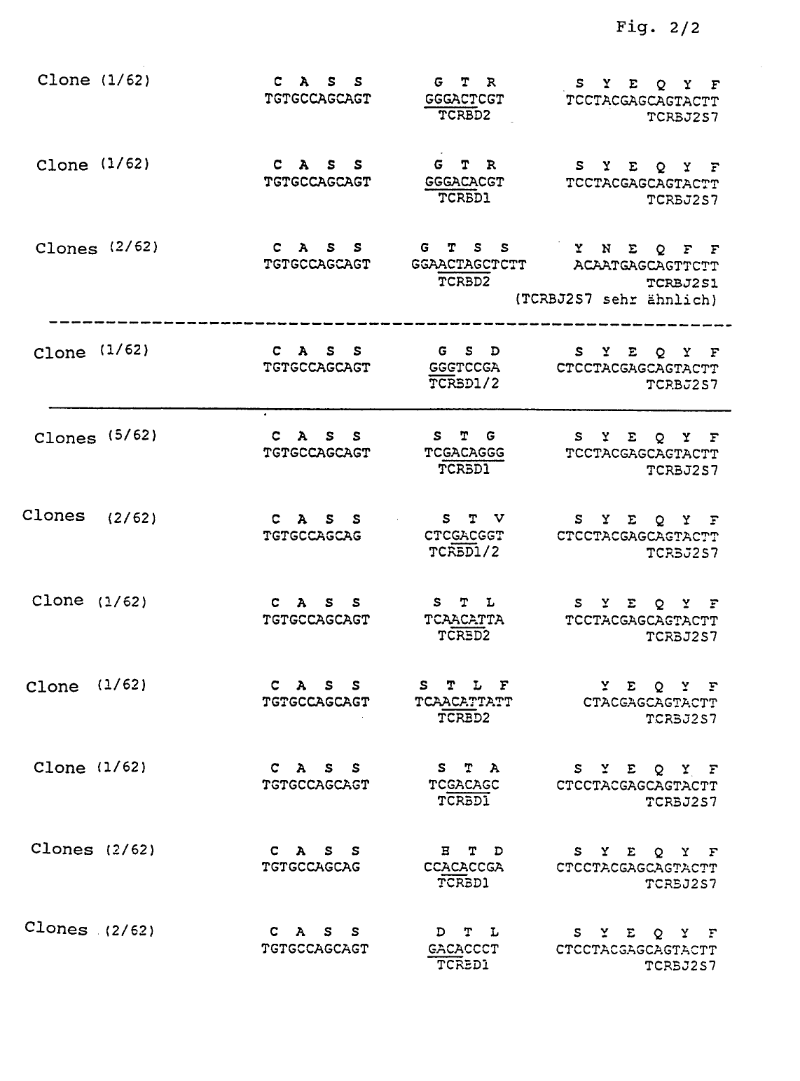

Furthermore it was found in the in situ sequencing that ca. 70% of all sequences of the β chain correspond to the amino acid sequences shown in the sequence protocols 4, 10, 12, 14, 16, 18 and 20. An overview of the CDR3 sequences of the β chain identified in the in situ sequencing is shown in FIG. 2.

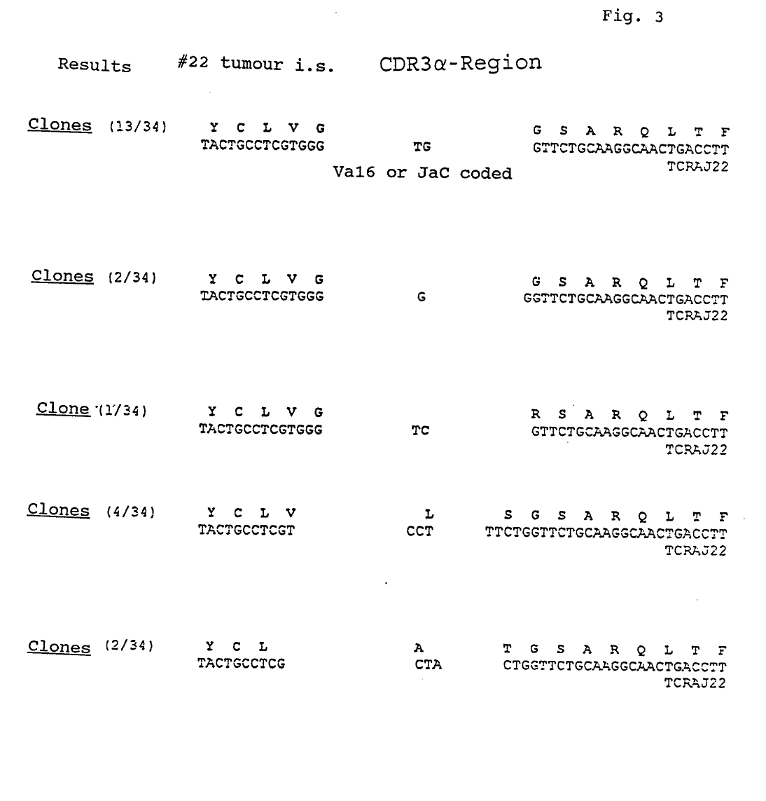

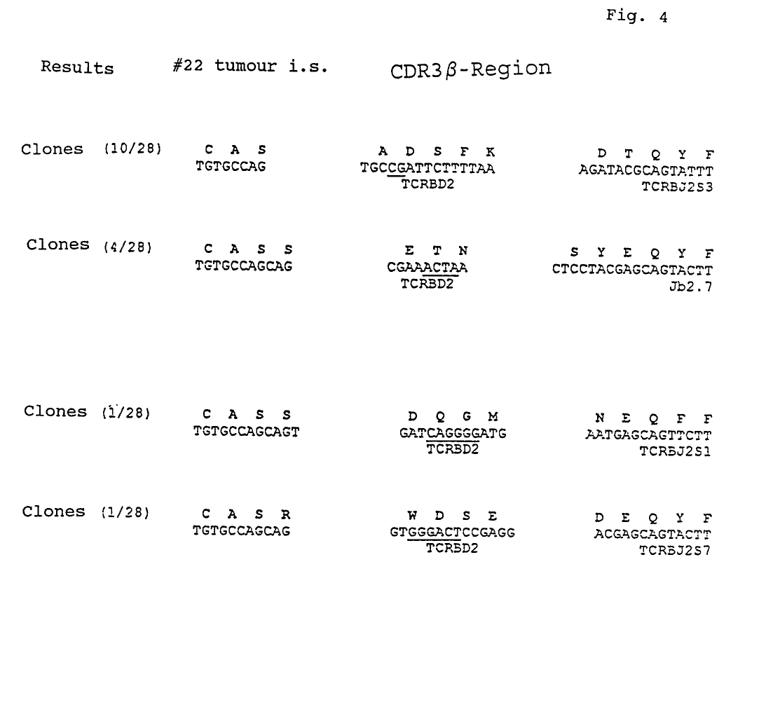

In a control experiment TIL from another patient with the HLA-A*0201 allele were analysed by in situ sequencing. It was found that the CDR3α regions of 15 and 4 of the total of 34 examined T cell species contained the amino acid sequences shown in SEQ ID NO. 2 and SEQ ID NO. 6. An overview of the relevant CDR3α sequences and their frequency is shown in FIG. 3. An overview of the results which were obtained when sequencing the CDR3 regions of the β chain is shown in FIG. 4.

Hence a first aspect of the present invention concerns a nucleic acid which codes for the α chain of a human T cell receptor, a functional derivative or a fragment thereof and comprises a CDR3 region composed of a combination of a Vα20 gene segment and a Jα22 gene segment. The length of the amino acid section coded by this CDR3 region is 11-14 amino acids and preferably 12 or 13 amino acids. The CDR3 region particularly preferably codes for one of the amino acid sequences shown in the sequence protocols SEQ ID NO. 2, 6 and 8, a sequence that is at least 80% and in particular at least 90% identical to this or a sequence which codes for an amino acid sequence with an equivalent recognition specificity for the peptide component of the T cell receptor ligand.

A further aspect of the present invention is a nucleic acid which codes for the a chain of a human T cell receptor, or for a functional derivative or a fragment thereof and comprises a CDR3 region selected from:

(a) a nucleotide sequence coding for the amino acid sequence

Y C L (X₁ . . . Xₙ) S A R Q L T F (I)

in which X₁ . . . Xₙ represents a sequence of 3-5 amino acids,

(b) a nucleotide sequence which codes for an amino acid sequence which is at least 80% and in particular at least 90% identical with the amino acid sequence from (a) or

(c) a nucleotide sequence which codes for an amino acid sequence with an equivalent recognition specificity for the peptide component of the T cell receptor ligand.

The amino acid sequence X₁ . . . Xₙ is preferably selected from the group comprising the amino acid sequences VGG, VLSG, ATG, VSG, DSG, VVSG, ALAG, APSG and VGR. The amino acid sequence X₁ . . . Xₙ is particularly preferably selected from the amino acid sequences VGG, VLSG and ATG.

A particular feature of the tumour-specific CDR3α regions of the invention is a length of 12-13 amino acids and a common sequence motif. Thus if the sequence X₁ . . . Xₙ has a length of 3 amino acids X₁ is preferably V or A, X₂ is preferably T, G or S and X₃ is preferably G. If the length of the sequence X₁ . . . Xₙ is 4 amino acids then preferably X₁=V or A, at least one of X₂ or X₃ is T or S and X₄=G.

A sequencing of the β chains from both patients that were examined yielded a combination of the gene segments Vβ22, Dβ1 or Dβ2 and Jβ2.7 for the first patient and a combination of the gene segments Vβ22, Dβ1 or Dβ2 and Jβ2.1, Jβ2.3 or Jβ2.7 for the second patient.

Hence a further aspect of the present invention is a nucleic acid which codes for the β chain of a human T cell receptor, or for a functional derivative or a fragment thereof and comprises a CDR3 region which is composed of a combination of a Vβ22 gene segment of a Dβ1 or Dβ2 gene segment and of a Jβ gene segment in particular of a Jβ2.1, Jβ2.3 or Jβ2.7 gene segment.

The length of the amino acid section coded by this CDR3β region is 12-14 amino acids, preferably 13 amino acids. Furthermore this CDR3β region preferably contains a common sequence motif i.e. X-T or S-X-S in which X represents an arbitrary amino acid and T or S particularly preferably denote T. A total of 70% of the examined T cell receptors have such a sequence pattern.

Yet a further aspect of the present invention is a nucleic acid which codes for the β chain of a human T cell receptor, or for a functional derivative or a fragment thereof and comprises a CDR3 region which is selected from:

(a) a nucleotide sequence coding for the amino acid sequence

C A (X′₁ . . . X′ₙ) Y/D E Q Y F (II)

in which X′₁ . . . X′ₙ represents a sequence of 5-7 amino acids,

(b) a nucleotide sequence coding for the amino acid sequence

C A (X″₁ . . . X″ₙ) N E Q F F (III)

in which X″₁ . . . X″ₙ represents a sequence of 5-7 amino acids,

(c) a nucleotide sequence coding for the amino acid sequence

C A (X′″₁ . . . X′″ₙ) D T Q Y F (IV)

in which X′″₁ . . . X′″ₙ represents a sequence of 5-7 amino acids,

(d) a nucleotide sequence which codes for an amino acid sequence that is at least 80% and in particular at least 90% identical with an amino acid sequence from (a), (b) or/and (c), or

(e) a nucleotide sequence which codes for an amino acid sequence with an equivalent recognition specificity for the peptide component of the T cell receptor ligand.

The amino acid sequence X′₁ . . . X′ₙ is preferably selected from the group comprising SSETNS, SSETSS, TSGTAS, RSGTGS, SSGTDS, SSGTRS, SSGSDS, SSSTGS, SSSTVS, SSSTLS, SSSTLF, SSSTAS, SSHTDS, SSDTLS and SRWDSE. The amino acid sequence X′₁ . . . X′ₙ particularly preferably represents SSETNS, SSGTDS, TSGTAS or RSGTGS. The amino acid sequence Xi″₁ . . . X″ₙ preferably denotes SSGTSSY or SSDQGM. The amino acid sequence X′″₁ . . . X′″ₙ preferably denotes SADSFK.

Within the sense of the present invention the term “functional derivative of a chain of a human T cell receptor” is understood as a polypeptide which comprises at least one CDR3α or/and CDR3β region as defined above and together with the respective complementary chain of the human T cell receptor (or a derivative of such a chain) can form a T cell receptor derivative which has an equivalent recognition specificity for a peptide ligand presented by a MHC molecule to that of the non-derivatized T cell receptor. Such a T cell receptor has a binding constant of at least 10⁻⁴ l/mol, preferably 10⁻⁴ to 10⁻⁵ l/mol for the presenting peptide ligand.

Functional derivatives of chains of a human T cell receptor can for example be prepared by deletion, substitution or/and insertion of sections of the gene coding for the respective polypeptide by means of recombinant DNA techniques. The preparation of recombinant T cell receptor chains is for example described in Blank et al. (1993), Eur. J. Immunol. 23, 3057-3065; Lin et al. (1990) Science 249: 677, Gregoire et al. (1991), Proc. Natl. Acad. Sci. USA, 88: 8077; Kappes and Tonegawa (1991), Proc. Natl. Acad. Sci. USA 88: 10619 and Ward (1991), Scand. J. Immunol. 34: 215. Explicit reference is herewith made to these literature citations.

Particular preferred functional derivatives of T cell receptor chains or T cell receptors are single chain T cell receptors which can for example be composed of the variable domains of the α and β chain and a constant domain. The preparation of such constructs is described by Chung et al. (1994), Proc. Natl. Acad. Sci. USA 91: 12654-12658. A further preferred example of functional derivatives are soluble TCR fragments which can be prepared as separate polypeptides or as single chain polypeptides cf. e.g. Hilyard et al. (1994), Proc. Natl. Acad. Sci. USA 91; 9057-9061. Explicit reference is also made to the disclosure in these literature citations.

A further subject matter of the present invention is a vector which contains at least one copy of a nucleic acid according to the invention. This vector can be a prokaryotic vector or a eukaryotic vector. Examples of prokaryotic vectors are plasmids, cosmids and bacteriophages. Such vectors are described in detail in Sambrook et al., Molecular Cloning. A Laboratory Manual, 2nd Edition (1989), Cold Spring Harbor Laboratory Press, in chapters 1-4. The prokaryotic vector is preferably a plasmid.

On the other hand the vector can also be a eukaryotic vector e.g. a yeast vector, a plant vector (bacolovirus) or a mammalian vector (a plasmid vector or a viral vector). Examples of eukaryotic vectors are described in Sambrook et al., Supra, chapter 16 and Winnacker, Gene and Klone, “Eine Einführung in die Gentechnologie” (1985), VCH “Verlagsgesellschaft” in particular in chapters 5, 8 and 10.

Yet a further subject matter of the invention is a cell which expresses a nucleic acid according to the invention or a cell which is transformed with a nucleic acid according to the invention or with a vector according to the invention. The cell can be a prokaryotic cell (e.g. a gram-negative bacterial cell, in particular E. coli) or a eukaryotic cell (e.g. a yeast, plant or mammalian cell). Examples of suitable cells and methods for introducing the nucleic acid according to the invention into such cells may be found in the above literature references.

A further subject matter of the present invention is a polypeptide which is coded by a nucleic acid according to the invention. The polypeptide particularly preferably contains the variable domain of the α or/and β chain of a human T cell receptor.

A polypeptide is particularly preferred which has T cell receptor properties and is composed of a TCRα chain or a functional derivative thereof as well as a TCRβ chain or a functional derivative thereof as subunits. The polypeptide can be composed of two separate chains or be present as a single chain polypeptide. In addition the polypeptide may also be present in an oligomerized form in which at least 2 and preferably 2-10 TCRα and TCRβ chains are linked together. The linkage can for example be achieved by means of bifunctional chemical linkers.

Yet a further subject matter of the present invention is an antibody against a polypeptide according to the invention which is directed towards a region of the polypeptide which is responsible for recognizing the peptide ligand. This antibody can be a polyclonal antiserum, a monoclonal antibody or a fragment of a polyclonal or monoclonal antibody (e.g. a Fab, F(ab)₂, Fab′ or F(ab′)₂ fragment). The antibody is preferably directed towards a CDR3 region of the polypeptide or an area thereof. Such antibodies can be obtained by well-known methods by immunizing an experimental animal with a peptide or polypeptide which contains a CDR3 region according to the invention and isolating the resulting antibodies from the experimental animal. Monoclonal antibodies can be obtained by fusing an antibody-producing B cell of the experimental animal with a leukemia cell according to the method of Köhler and Milstein or further developments thereof. Specific examples of the production of such antibodies can be found in Choi et al. (1991), Proc. Natl. Acad. Sci. USA 88: 8357-8361 and Zumla et al. (1992), Hum. Immunol. 35: 141.

Yet a further subject matter of the present invention is a T cell which contains a T cell receptor according to the invention. Such T cells can be isolated from patients with kidney cell carcinoma and then be expanded in vitro. For this the peripheral mononuclear blood cells of a patient can for example be produced by stimulation with suitable antigens and subsequent restimulation for example with an irradiated autologous lymphoblastoid cell line, tumour cells, lymphoblastoid cells plus antigens or autologous peripheral blood lymphocytes plus antigen. Further methods for obtaining T cells according to the invention are described below.

The invention also concerns a pharmaceutical composition which contains a nucleic acid, a polypeptide, a peptide ligand capable of binding to the polypeptide optionally in association with a corresponding MHC molecule, an antibody or a cell as described above as active components optionally together with other active components as well as common pharmaceutical auxiliary substances, additives or carrier substances. Examples of other active components are accessory stimulating components e.g. cytokines such as IL-2 and IL-4.

The pharmaceutical composition can be used to produce a diagnostic or therapeutic agent. Examples of diagnostic applications are the diagnosis of tumour diseases or a predisposition for tumour diseases. A further preferred diagnostic application is the monitoring of the disease course in a tumour disease e.g. after chemotherapy or a surgical operation.

The use of the pharmaceutical composition as a diagnostic agent preferably comprises the detection of a T cell subpopulation which expresses a polypeptide according to the invention as a T cell receptor. The detection of this T cell receptor can for example be achieved at the nucleic acid level e.g. by a nucleic acid hybridization assay optionally with a prior amplification. On the other hand the detection can also be carried out at the protein level by an immunoassay using antibodies that react specifically with the T cell receptor. In addition it is also possible to detect the T cells for example by means of a test for binding to specific peptide ligands or in an activity test in which the specific cytotoxic action of the T cells or the release of cytokines such as TNF or IFNγ is determined.

Furthermore the pharmaceutical composition according to the invention can also be used therapeutically in particular for the prevention or therapy of a tumour disease e.g. of a kidney cell carcinoma. This therapeutic application can for example be based on the fact that T cells which express the tumour specific T cell receptor are stimulated to grow in vitro or in vivo. The growth stimulation in vivo can for example be achieved by administering the peptide ligand of the T cell receptor or/and the whole molecule from which the peptide ligand is derived or a fragment thereof. Furthermore the growth stimulation in vivo can also be accomplished by administering an antibody which specifically activates the T cell receptor by binding e.g. a monoclonal antibody or a monoclonal antibody fragment.

On the other hand the growth stimulation of the T cells can also be carried out in vitro for example by isolating specific T cells from the patient, in vitro expansion and subsequent administration of the expanded T cells as a tumour vaccine. T cells which express a tumour specific T cell receptor are isolated from a patient preferably by contacting a sample from the patient which contains T cells, e.g. a blood sample and preferably a sample derived from the tumour tissue, with an agent which specifically binds to the CDR3 region of the T cell receptor, identifying the T cells which react with the agent and optionally separating them from other T cells. The agent that binds to the CDR3 region of the T cell receptor is preferably selected from the peptide ligand of the T cells, a peptide ligand-MHC complex or/and an anti-TCR antibody. Optionally the in vitro expansion can additionally be carried out in the presence of costimulatory factors such as anti-CD28 antibodies. In order to facilitate separation of the desired T cell subpopulation, the agent is preferably used in an immobilized or immobilizable form.

The isolation of T cells which express a tumour specific T cell receptor can, however, also be achieved in another manner e.g. by introducing nucleic acid sequences which code for the T cell receptor into a T cell line, preferably a cytotoxic T cell line. The T cell receptor is then expressed in this transfected T cell line. In this manner it is possible to obtain T cells in large amounts which express a tumour specific T cell receptor.

Yet another method for isolating T cells which express a tumour specific T cell receptor is to introduce nucleic acid sequences which code for the T cell receptor into the germ line of an animal and to isolate the T cells from the resulting transgenic animal or its descendants. Transgenic mice are preferably produced. Furthermore it is preferred that the transgenic mice also express the human CD8 molecule or/and the human HLA-A*0201 molecule in addition to the T cell receptor.

Hence a further subject matter of the present invention is also a transgenic animal which has T cells that express a tumour specific T cell receptor. This transgenic animal is preferably a rodent in particular a mouse.

Finally the invention also concerns a method for the identification of peptide ligands of a T cell receptor according to the invention. This method preferably comprises the steps:

(a) isolating RNA from tumour tissue,

(b) converting the RNA into double-stranded cDNA molecules,

(c) introducing the cDNA molecules into host cells to obtain a cDNA bank,

(d) transfecting eukaryotic recipient cells with aliquots of the cDNA bank in which (i) there is a cotransfection with HLA-A*0201 DNA or (ii) HLA-A*0201-positive recipient cells are used,

(e) testing the transfected recipient cells for their ability to stimulate T cells to for example proliferate or to secrete cytokines such as TNF in which case it is possible for example to examine the lysis of TNF-sensitive cells,

(f) identifying a cDNA sequence which codes for the antigen which contains the peptide ligand and

(g) identifying the sequence of the peptide ligand.

The invention is further elucidated by the following examples, figures and sequence protocols.

SEQ ID NO. 1: shows the nucleotide sequence of the TCRα chain of a T cell receptor according to the invention in which bp 55-324/325 codes for the TCR-Vα20 gene segment, bp 325/326 codes for the TCR Jα22 gene segment, bp 381-804 codes for the TCR-Cα gene segment and bp 805-1341 represent a 3′ untranslated region,

SEQ ID NO. 2: shows the amino acid sequence of the nucleotide sequence shown in SEQ ID NO. 1,

SEQ ID NO. 3: shows the nucleotide sequence of the TCRβ chain of a T cell receptor according to the invention in which bp 1-63 are nucleotides, bp 346-349 code for the TCR-Dβ2 gene segment, bp 350 is an N-nucleotide, bp 351-398 code for the TCR-Jβ2.7 gene segment and bp 399-936 code for the TCR-Cβ gene segment,

SEQ ID NO. 4: shows the amino acid sequence of the nucleotide sequence shown in SEQ ID NO. 3,

SEQ ID NO. 5 and 6 show nucleotide and amino acid sequences of the CDR3α region of a T cell receptor according to the invention

SEQ ID NO. 7 and 8: show nucleotide and amino acid sequences of the CDR3α region of a T cell receptor according to the invention

SEQ ID NO. 9 and 10: show nucleotide and amino acid sequences of the CDR3β region of a T cell receptor according to the invention

SEQ ID NO. 11 and 12: show nucleotide and amino acid sequences of the CDR3β region of a T cell receptor according to the invention

SEQ ID NO. 13 and 14: show nucleotide and amino acid sequences of the CDR3β region of a T cell receptor according to the invention

SEQ ID NO. 15 and 16: show nucleotide and amino acid sequences of the CDR3β region of a T cell receptor according to the invention

SEQ ID NO. 17 and 18 show nucleotide and amino acid sequences of the CDR3β region of a T cell receptor according to the invention

SEQ ID NO. 19 and 20: show nucleotide and amino acid sequences of the CDR3β region of a T cell receptor according to the invention

SEQ ID NO. 21 shows the nucleotide sequence of the TCRα-specific primer P-CαST,

SEQ ID NO. 22 shows the nucleotide sequence of the TCRβ-specific primer P-CαST

FIG. 1 shows nucleotide and amino acid sequences of the CDR3α regions from tumour-specific TCR which have been determined by in situ sequencing of T cells of patient 26,

FIG. 2 shows nucleotide and amino acid sequences of CDR3β regions of tumour-specific TCR which have been determined by in situ sequencing of T cells of patient 26,

FIG. 3 shows nucleotide and amino acid sequences of CDR3α regions of tumour-specific TCR which have been determined by in situ sequencing of T cells of patient 22,

FIG. 4 shows nucleotide and amino acid sequences of CDR3β regions of tumour-specific TCR which have been determined by in situ sequencing of T cells of patient 22,

### EXAMPLE 1

Analysis of T cell Receptors in HLA-A2 Patients With Kidney Cell Carcinoma

Cytotoxic CD8⁺T cells were identified in kidney cell patient 26 which lysed autologous tumour cells with a HLA-A2 restricted mechanism. The T cells have a high tumour specificity since short-term cultures of normal kidney cells are not recognized. The determinants recognized by the TIL of patient 26 were also found on other tumours of patients which carry the HLA-A2 gene in particular the widespread HLA-A*0201 allele. Normal kidney cells of these patients were not lysed. These results show that the kidney carcinoma cells of patient 26 express a tumour determinant i.e. a tumour-associated peptide/HLA-A2 complex which is also present on the tumours of other patients.

Total RNA is isolated from T cells in order to identify and sequence tumour-specific TCR. For this the cells in suspension are washed with PBS and the cell pellet is resuspended with 0.2 ml RNazol-B per 1×10⁶ cells. 2 ml RNazol-B per 100 mg tissue is added to extract the RNA from the tissue. After mechanically resuspending the lysates several times and optionally adding yeast tRNA as a carrier matrix, the RNA is extracted by adding 0.2 ml chloroform per 2 ml homogenate, subsequently mixing for 15 sec. and storing for 5 minutes on ice.

After a centrifugation step at 12,000 g for 15 min at 4° C., the aqueous phase is removed and transferred into a new reaction vessel. The first precipitation of the RNA is carried out by adding an identical volume of isopropanol and subsequently storing for at least 15 min at 4° C. After centrifuging for 15 min at 12,000 g and 4° C., the RNA is obtained as a white pellet at the bottom of the vessel.

After discarding the supernatant, the RNA pellet is purified of salts by briefly mixing in 75% ethanol. After centrifuging (7500 g, 4° C., 8 min), the pellet is dissolved in 175 μl water treated with diethylpyrocarbonate (DEPC) and precipitated again with 500 μl ethanol and 75 μl 2 M NaCl for at least 1 h at −20° C. The centrifugation and washing steps after the second precipitation are carried out as described for the first precipitation. After drying the pellets in air, the RNA is resuspended in H₂O-DEPC or 0.5% SDS, pH 6.5 to 7.0 or 1 mM EDTA, pH 7.0.

Subsequently cDNA is synthesized from the RNA by reverse transcription. For this 3 μg total RNA is incubated for 10 minutes at 55° C. with 30 ng P-CαST (a specific primer for the TCRα chain with the sequence 5′-CAC TGA AGA TCC ATC ATC TG-3′ shown in SEQ ID NO. 21) and 30 ng P-CβST (a specific primer for the TCRβ chain with the sequence 5′-TAG AGG ATG GTG GCA GAC AG-3′ shown in SEQ ID NO. 22) in a reaction volume of 10 μl. Afterwards 38 μl RAV-2-RT buffer (100 mM Tris-HCl pH 8.3; 140 mM KCl; 10 mM MgCl₂; 2 mM dithiothreitol, 0.1 mM of each dNTP), 1 μl (0.75 U) rRNasin and 1 μl (18 U) reverse transcriptase are added by pipette. The reverse transcription is carried out for 1 h at 42° C., followed by a denaturation step at 68° C. for 5 min. It is stored until use at −20° C.

Subsequently a polymerase chain reaction is carried out. The primer can be biotinylated in order to enable the PCR products to be subsequently purified by coupling to a magnetic particulate solid phase (streptavidin-coated beads).

The PCR is carried out using a thermostable DNA polymerase and the following reaction scheme:

95° C. 5 min. predenaturation (only at the beginning)

95° C. 30 sec DNA denaturation

56° C. 1 min annealing

72° C. 1 min extension

72° C. 10 min filling up all single strands in the reaction solution (only at the end).

The number of reaction cycles in the PCR is usually 30.

The PCR fragments obtained in this manner are sequenced.

When the cytotoxic T cells from patient 26 are cultured and periodically restimulated over a period of 62 and 74 days respectively, a uniform CD8⁺T cell clone is obtained. The nucleotide and amino acid sequence of the TCRα chain of this T cell clone from patient 26 are shown in SEQ ID NO. 1 and 2. The nucleotide and amino acid sequence of the TCRβ chain are shown in SEQ ID NO. 3 and 4.

When the tumour infiltrating lymphocytes from patient 26 were only cultured for 24 days, the T cell clone was not found to be homogeneous but rather a mixture of several T cell species. The CDRα regions of these T cell species contained a total of two further sequences (SEQ ID NO. 5 and 6 and 7 and 8) in addition to the amino acid sequence shown in SEQ ID NO. 2. In addition to the amino acid sequence shown in SEQ ID NO. 4, the CDR3β regions contained further closely related sequences (SEQ ID NO. 9 and 10, 11 and 12, 13 and 14, 15 and 16, 17 and 18 and 19 and 20).

Furthermore the T cells of patient 26 were sequenced in situ i.e. sequenced without prior culturing. In this process a series of individual sequences was obtained for the CDR3α region which are shown in FIG. 1. Circa 60% of all sequences of the α chain correspond to the sequences previously described. A further 20% correspond to very similar sequences.

Also in the case of the CDR3 regions of the β chain it was found that a total of 70% of the examined T cells of patient 26 had a very similar sequence pattern (FIG. 2).

Peripheral blood samples from patient 26 were analyzed for T cell receptors which have features of tumour-specific T cell receptors over 4 years in all. It was found that such sequences only occurred with a frequency of about 1/150,000 T cells.

Cytotoxicity investigations showed that the tumour-specific T cells isolated from patient 26 could also lyse tumour cells of patient 22 which also carry the HLA-A*0201 allele. Tumour infiltrating T cells from patient 22 could in turn lyse tumour cells from patient 26. A sequencing of the T cell receptors from patient 22 yielded the results shown in FIG. 3 for the CDR3α region and in FIG. 4 for the CDR3β region.

### EXAMPLE 2

Expression of T cell Receptors

2.1 Expression of Tumour Specific T cell Receptors in Human or Murine T cell Lines

The nucleic acid sequences identified in example 1 which code for tumour specific TCRα and β chains are cloned into eukaryotic human and murine expression vectors. The human expression vector is described in Chung et al. (Proc. Natl. Acad. Sci. USA 92 (1995): 3712-3716). The murine vectors are described in Gabert et al. (Cell 50 (1987: 545-554) and Gregoire et al. (Proc. Natl. Acad. Sci. USA 88 (1991): 8077-8081).

The TCR DNA can either be cloned from rearranged genomic DNA or from cDNA. Basically two cloning strategies are available: firstly the isolation of very long TCRα and β DNA fragments from the genome of mature T cells which contain several Kb long 5′ flanking sequences with all regulatory elements required for expression. Alternatively vectors can be selected which already contain the natural 5′ regulatory elements and in which only short fragments coding for the variable regions have to be cloned in (Kouskoff et al. J. Immunol. Methods 180 (1995): 273-280). In the latter method the sequence of the variable region (including the leader sequence) is examined for mistakes by sequencing after amplification by means of specific PCR and subsequently introduced into the vector after digestion with appropriate restriction endonucleases.

The PCRα and β chains can either be cloned into a common vector or into two different vectors. Each of the vectors used contains a selection marker which enables the positive selection of successfully transfected cells after transfection of the recipient cells with the recombinant plasmid. Preferred selection markers are for example the gene for neomycin resistance (neo) or the gene for xanthine-guanine-phosphoribosyl-transferase (GPT).

2.2 Expression of Functional T cell Receptors as Single Chain Constructs

Similarly to antibodies it is possible to express TCR as single chain constructs in eukaryotic cells (Chung et al., Proc. Natl. Acad. Sci. USA 91 (1994): 12654-12658). In this method a construct is prepared which also contains the constant domain of the β chain in addition to the variable domains of the TCRα and β chain. The individual domains are amplified by means of PCR as described in example 1 after isolation of the corresponding RNA and reverse transcription. In this process suitable restriction cleavage sites are inserted at the ends of the amplification products. The individual fragments are then ligated together as follows in a eukaryotic expression vector (e.g. pBJ-Neo) which carries a positive selection marker: the variable TCRα and β domains comprising leader, V-(D)- and J exon are separated by a linker sequence e.g. a DNA fragment coding for the amino acid sequence (GGGGS)₃. The exon for the constant TCRβ domain is ligated directly to the variable β domain.

Alternatively coding sequences for a GPI anchor (Lin et al., Science 249 (1990): 677-679) or for example the transmembrane part and the intracellular domain of the CD3ζ chain (Engel et al., Science 256 (1992): 1318-1321) can be ligated to the 3′ end of this construct. After transfection of these constructs in eukaryotic cells, the former enables the production of soluble TCR molecules which can be used as an immunogen to produce antibodies. The latter enables the functional analysis of the construct in biological systems.

2.3 Production of Soluble Human TCR Fragments in E. coli

Large amounts of soluble TCR fragments can be produced in E. coli as single chain polypeptides (Hilyard et al., Proc. Natl. Acad. Sci. USA 91 (1994): 9057-9061).

For this various genes or gene fragments are cloned into an inducible prokaryotic vector e.g. pUC19. The fragments to be ligated are reamplified by means of specific PCR in the process of which suitable restriction cleavage sites are added.

The following fragments are cloned into the vector in the order shown:

1. A prokaryotic signal sequence e.g. the pelB-leader sequence from the pectate lyase gene of Erwinia carolovora (Ward et al., Nature 341 (1989): 8646-8650) which causes a secretion of the polypeptide into the periplasm of the host bacterium.

2. The variable PCRα and β chain fragments from a tumour-specific TCR. These fragments are preferably separated by a linker e.g. the linker shown in example 2.2. which improves the solubility and the flexibility of the synthesized molecule.

3. A nucleotide sequence coding for a tail made of several e.g. 6 histidine residues which enables the recombinant polypeptide to be isolated by affinity chromatography e.g. by nickel chelate chromatography.

### EXAMPLE 3

Production of Antibodies Against Tumour-specific T cell Receptors

Mice are immunized with the appropriate antigen to produce antisera or monoclonal antibodies against tumour-specific TCR. The immunization is carried out according to the protocols given by Harlow, E. and David, C., Antibodies. A Laboratory Manual, Cold Spring Harbor Laboratory, 1988. TCR expressing cells (example 2.1) or soluble TCR (example 2.2 or example 2.3) can for example be selected as antigens.

Alternatively the soluble TCR used for the immunization can also be produced as chimeric proteins which are composed of a variable TCR region, a truncated constant TCR region and a constant immunoglobulin region (cf. e.g. Gregoire et al. (1991), Supra). For this the specific variable TCRα and β regions are each cloned into a plasmid which already contains the first exon, a corresponding C region and an IgGk domain. Both plasmids additionally contain a positive selection marker and the regulatory elements required for correct expression. Both plasmids are then used to transfect a mouse myeloma cell line which does not express endogenous heavy and light Ig chains. After the transfection is completed both chimeric chains are synthesized and preferentially secreted as heterodimers.

Alternatively a TCR protein antigen for immunizing mice can be constructed as follows: A human V gene segment is fused to a TCR gene segment composed of (D), J and C gene segments from a mouse T cell hybridoma i.e. the gene segments are cloned in this order into a eukaryotic expression vector (Choi et al., Proc. Natl. Acad. Sci. USA 88 (1991): 8357-8361). The human sequence is obtained from the corresponding cDNA by means of PCR by amplifying the V region. Such constructs are then used to transfect mouse T cell hybridomas which provide all components apart from the corresponding transfected chains. Since the plasmids also code for selection markers, transfectants can be positively selected by an appropriate medium. Since these transfectants represent mouse T cells which express a human V region, mice that are immunized with such cells only produce antibodies against this foreign human sequence.

### EXAMPLE 4

Identification of the Peptide Ligands of Tumour Specific T cells

Poly-A⁺mRNA is isolated from a kidney cell carcinoma line using a commercial kit (Fastrack/Invitrogen) and converted into double-stranded cDNA using the Superscript Choice System kit (Gibco) using a NotI/Oligo-dT primer for the first strand synthesis. The cDNA is ligated with BstXI adaptors and cleaved with NotI. High molecular size fractionated cDNA is selected and cloned into the vector pcDNAI/Amp (Invitrogen) cleaved with BstXI and NotI.

E. coli DH5α cells are transformed by electroporation with the recombinant plasmids and selected with ampicillin. The cDNA bank obtained in this manner is divided into 1500 pools each comprising approximately 100 clones. Each pool is amplified to saturation and the plasmid DNA is isolated from this by alkaline lysis without phenol extraction.

In each case approximately 100 ng plasmid DNA of a pool is transfected together with 50 ng plasmid DNA of the same vector which carries the HLA-A*0201-cDNA (gene bank, ACC No.: M32322, K02883, M84379, X02457) into 15000 COS7 cells according to the DEAE-dextranchloroquine method. Alternatively the COS7 cells can also be transfected with the HLA-A*0201 DNA and the stable transfectants obtained in this manner can be used as recipient cells.

24-48 hours after transfection the COS7 cells are tested for their ability to stimulate the release of TNF by tumour specific cytotoxic T cells (CTL). A test is carried out in each case with 200 pools i.e. 200 independent transfections of COS7 cells.

For this 3000 CTL are added to the wells of microtitre plates containing COS7 transfectants. After 18 hours the supernatant of the medium is collected and its TNF content is determined using an activity test in which TNF sensitive cell lines such as the mouse fibroblast cell lines WEHI 164 or L929 are lysed by TNF. Viable cultures can be distinguished from lysed cells by a colorimetric test using 3-(4,5-dimethylthiazol-2-yl)2,5-diphenyltetrazolium bromide (MTT).

A new cycle of COS7 transfection was carried out for each positive microculture in which smaller pools of bacteria from the original pool containing a total of 100 clones were used in each case. This procedure is repeated until a single plasmid is identified which can induce the TNF release from the specific TCL after co-expression with HLA-A*0201 cDNA in COS7 cells.

The sequence of the plasmid insertion is determined by standard methods. The confirmation that this sequence codes for the tumour peptide is achieved by transfecting normal human HLA-A*0201 cells which are not lysed by the tumour specific CTL. These cells are sensitive for a lysis after transfection with the corresponding cDNA. Furthermore the tumour specific expression of the identified cDNA is determined by Northern blot using the cDNA as a probe. This probe is used for hybridization to mRNA from various tumour cell lines of normal tissue samples.

The tumour specific peptide can be identified by various methods. The corresponding protein sequence is derived from the cDNA sequence and screened for binding motifs which had been identified in other HLA-A*0201 binding peptides. Synthetic peptides which overlap with potential HLA-A*0201 binding regions are then tested for their ability to activate CTL after incubation with HLAA*0201 cells. Alternatively overlapping peptides of 8-9 amino acids in length can be produced by synthesis and tested in a similar manner.

### EXAMPLE 5

Production of Transgenic Mice

Total RNA is isolated from a specific T cell clone and cDNA is synthesized by reverse transcription (cf. example 1). Using primers specific for the V region, TCR-cDNA for the Vα and Vβ regions is amplified and cloned into TCR gene cassettes which contain constant regions and the necessary regulation elements for expression. Separate cassettes for TCRα and TCRβ sequences are known which each carry a different selection marker (Kouskoff et al., (1995), Supra).

Fertilized mouse oocytes are simultaneously microinjected with DNA from the TCRα as well as from the TCRβ cassettes. The injected oocytes are transferred back into female mice (Mellor, A. L., Transgenesis and the T cell receptor. in: T cell receptors (1995), J. I. Bell, M. J. Owen and E. Simpson, eds. pp 194-223, Oxford University Press, Oxford, New York, Tokyo).

The introduction of productively rearranged TCR genes in the mouse has a major influence on the TCR repertoire since rearranged TCR foreign genes prevent the further rearrangement of endogenous TCR genes. Consequently nearly all thymocytes and T cells express the heterologous TCR clonotype so that the TCR repertoire in such mice is essentially monoclonal.

Transgenic mice are identified by genotype analysis using probes which are specific for the DNA contained in the foreign gene that does not occur in the mouse genome. This can either be carried out by Southern blot hybridization or preferably by PCR.

Transgenic descendants of the mice are obtained by crossing with non-transgenic mice of a suitable strain, typing the descendants and using them for further crossing.

1. Nucleic acid which codes for the α chain of a human T cell receptor, or for a functional derivative or a fragment thereof and which comprises a CDR3 region formed from a combination of a Vα20 and Jα22 gene segment.

2. Nucleic acid which codes for the a chain of a human T cell receptor, or for a functional derivative or a fragment thereof and comprises a CDR3 region selected from:
(a) a nucleotide sequence coding for the amino acid sequence
Y C L (X₁ . . . Xₙ) S A R Q L T F (I)
in which X₁ . . . Xₙ represents a sequence of 3-5 amino acids,
(b) a nucleotide sequence which codes for an amino acid sequence which is at least 80% identical with the amino acid sequence from (a), or
(c) a nucleotide sequence which codes for an amino acid sequence with an equivalent recognition specificity for the peptide component of the T cell receptor ligands.

3. Nucleic acid as claimed in claim 2,
wherein
the amino acid sequence X₁ . . . Xₙ is selected from the group comprising the amino acid sequences VGG, VLSG, ATG, VSG, DSG, VVSG, ALAG, APSG and VGR.

4. Nucleic acid as claimed in claim 3,
wherein
the amino acid sequence X₁ . . . Xₙ is selected from the group comprising amino acid sequences VGG, VLSG and ATG.

5. Vector,
wherein
it contains at least one copy of a nucleic acid as claimed in one of the claims 1 to 4.

6. Cell,
wherein
it expresses a nucleic acid as claimed in one of the claims 1 to 4.

7. Cell,
wherein
it is transformed with a nucleic acid as claimed in one of the claims 1 to 4 or with a vector as claimed in claim 5.

8. Polypeptide,
wherein
it is coded by a nucleic acid as claimed in one of the claims 1 to 4.

9. Polypeptide as claimed in claim 8,
wherein
it comprises the variable domain of the a chain of a human T cell receptor.

10. Nucleic acid which codes for the β chain of a human T cell receptor, or for a functional derivative or a fragment thereof and comprises a CDR3 region formed from a combination of a Vβ22 gene segment, a Dβ1 or Dβ2 gene segment and a Jβ gene segment in particular a Jβ 2.1, Jβ2.3 or Jβ2.7 gene segment.

11. Nucleic acid which codes for the β chain of a human T cell receptor, or for a functional derivative or a fragment thereof and comprises a CDR3 region which is selected from:
(a) a nucleotide sequence coding for the amino acid sequence
C A (X′₁ . . . X′ₙ) Y/D E Q Y F (II)
in which X′₁ . . . X′ₙ represents a sequence of 5-7 amino acids,
(b) a nucleotide sequence coding for the amino acid sequence
C A (X″₁ . . . X″ₙ) N E Q F F (III)
in which X″₁ . . . X″ₙ represents a sequence of 5-7 amino acids,
(c) a nucleotide sequence coding for the amino acid sequence
C A (X′″₁ . . . X′″ₙ) D T Q Y F (IV)
in which X′″₁ . . . X′″ₙ represents a sequence of 5-7 amino acids,
(d) a nucleotide sequence which codes for an amino acid sequence that is at least 80% identical with an amino acid sequence from (a), (b) or/and (c), or
(e) a nucleotide sequence which codes for an amino acid sequence with an equivalent recognition specificity for the peptide component of the T cell receptor ligand.

12. Nucleic acid as claimed in claim 11,
wherein
the amino acid sequence X′₁ . . . X′ₙ is selected from the group comprising SSETNS, SSETSS, TSGTAS, RSGTGS, SSGTDS, SSGTRS, SSGSDS, SSSTGS, SSSTVS, SSSTLS, SSSTLF, SSSTAS, SSHTDS, SSDTLS and SRWDSE.

13. Nucleic acid as claimed in claim 12,
wherein
the amino acid sequence X′¹ . . . X′ₙ represents SSETNS, SSGTDS, TSGTAS or RSGTGS.

14. Nucleic acid as claimed in claim 11,
wherein
the amino acid sequence X″₁ . . . X″ₙ represents SSGTSSY or SSDQGM or the amino acid sequence X′″₁ . . . X′″ₙ represents SADSFK.

15. Vector,
wherein
it contains at least one copy of a nucleic acid as claimed in one of the claims 10 to 14.

16. Cell,
wherein
it expresses a nucleic acid as claimed in one of the claims 10 to 14.

17. Cell,
wherein
it is transformed with a nucleic acid as claimed in one of the claims 10 to 14 or with a vector as claimed in claim 15.

18. Polypeptide,
wherein
it codes for a nucleic acid as claimed in one of the claims 10 to 14.

19. Polypeptide as claimed in claim 18,
wherein
it comprises the variable domain of the β chain of a human T cell receptor.

20. Polypeptide,
wherein
it has T cell receptor properties and is composed of a polypeptide as claimed in claim 8 or 9 as well as a polypeptide as claimed in claim 18 or 19 as subunits.

21. Polypeptide as claimed in one of the claims 8, 9, 18, 19 or 20,
wherein
it is coupled to a labelling group or a toxin.

22. Polypeptide as claimed in one of the claims 8, 9, 18, 19, 20 or 21,
wherein
it is present in an oligomerized form.

23. Antibody against a polypeptide as claimed in one of the claims 8, 9, 18, 19, 20, 21 or 22 which is directed against a region which is responsible for recognizing the peptide ligand.

24. Antibody as claimed in claim 23,
wherein
it is directed towards a CDR3 region.

25. T cell,
wherein
it contains a T cell receptor as claimed in claim 20.

26. Pharmaceutical composition which contains as active component a nucleic acid as claimed in one of the claims 1 to 4 or 10 to 14, a polypeptide as claimed in one of the claims 8, 9 or 18 to 23, a peptide ligand against the polypeptide, an antibody as claimed in claim 23 or 24 or a cell as claimed in claim 6, 7, 16, 17 or 25 optionally together with other active components as well as common pharmaceutical auxiliary agents, additives or carrier substances.

27. Use of a pharmaceutical composition as claimed in claim 26 for the production of an agent for the diagnosis of tumour diseases or a predisposition for a tumour disease.

28. Use of a pharmaceutical composition as claimed in claim 26 for the production of an agent for monitoring the course of the disease in a tumour disease.

29. Use as claimed in claim 27 or 28,
wherein
the detection of T cells that express a polypeptide as claimed in claim 20 as the T cell receptor is carried out in a sample liquid by a nucleic acid hybridization assay, an immunoassay, a test for the binding of specific peptide ligands or a specific T cell activity test.

30. Use of a pharmaceutical composition as claimed in claim 26 for the production of an agent for the prevention or therapy of a tumour disease.

31. Use as claimed in claim 30,
wherein
the agent is suitable for the stimulation of the growth of T cells that express a polypeptide as claimed in claim 20 as a T cell receptor.

32. Use as claimed in claim 31,
wherein
the agent is suitable for growth stimulation of the T cells in vivo.

33. Use as claimed in claim 31 or 32,
wherein
the agent for growth stimulation comprises the peptide ligand of the T cell receptor or/and the entire molecule from which the peptide ligand is derived or a fragment thereof.

34. Use as claimed in claim 31 or 32,
wherein
the growth stimulation includes an antibody that specifically activates the T cell receptor.

35. Use as claimed in claim 31,
wherein
the growth stimulation is carried out by isolating specific T cells, in vitro expansion and subsequent administration of expanded T cells.

36. Use as claimed in one of the claims 27 to 35,
wherein
the tumour disease is a kidney cell carcinoma.

37. Process for the isolation of T cells that express a polypeptide as claimed in claim 20 as a T cell receptor,
wherein
a sample containing T cells is contacted with an agent that binds specifically to the CDR3 region of the T cell receptor, T cells that react with the agent are identified and optionally separated from other T cells.

38. Process as claimed in claim 37,
wherein
the agent is selected from the peptide ligand of T cells, a MHC peptide complex containing the peptide ligand or/and an anti-TCR antibody.

39. Process as claimed in claim 37 or 38 additionally comprising an in vitro expansion of T cells.

40. Process for the isolation of T cells which express a polypeptide as claimed in claim 20 as the T cell receptor,
wherein
nucleic acid sequences that code for the T cell receptor are introduced into a T cell line and are made to express therein.

41. Process for the isolation of T cells that express a polypeptide as claimed in claim 20 as the T cell receptor,
wherein
nucleic acid sequences which code for the T cell receptor are introduced into the germ line of an animal and the T cells are isolated from the resulting transgenic animal or descendants thereof.

42. Transgenic animal,
wherein
it expresses a polypeptide as claimed in claim 20 as the T cell receptor.

43. Method for the identification of peptide ligands of a T cell receptor as claimed in claim 20 comprising the steps:
(a) isolating RNA from tumour tissue,
(b) converting the RNA into double-stranded cDNA molecules,
(c) introducing the cDNA molecules into host cells to obtain a cDNA bank,
(d) transfecting eukaryotic recipient cells with aliquots of the cDNA bank wherein (i) cotransfection with HLA-A*0201 DNA is carried out or (ii) HLA-A*0201 positive recipient cells are used,
(e) testing the transfected recipient cells for their ability to stimulate T cells,
(f) identifying a cDNA sequence which codes for the antigen which contains the peptide ligand and
(g) identifying the sequence of the peptide ligand.

44. Method as claimed in claim 43,
wherein
step (e) comprises testing for the ability to lyse TNF-sensitive cells.
